(19) United States
(12) Patent Application Publication
(10) Pub. No.: US 2016/0206392 A1
(43) Pub. Date: Jul. 21, 2016

(54) Method and System for Reducing Patient Trauma or Anxiety During a Procedure or Surgery

(71) Applicant: Dipankar MUKHERJEE, Vienna, VA (US)

(72) Inventor: Dipankar MUKHERJEE, Vienna, VA (US)

(73) Assignee: Vibha AGARWAL, Burke, VA (US)

(21) Appl. No.: 15/000,253

(22) Filed: Jan. 19, 2016

Related U.S. Application Data
(60) Provisional application No. 62/105,859, filed on Jan. 21, 2015.

(51) Int. Cl. A61B 46/20; A61B 90/00; A61B 50/10

(52) U.S. Cl. CPC A61B 46/20; A61B 50/10; A61B 90/37

(57) ABSTRACT

A device for visually separating the portion of a patient's body being worked on by a medical professional during a surgery or procedure, includes a bracket for mounting to the bed supporting a patient undergoing a surgery or procedure, and a partition to be connected to the bracket for separating the portion of the patient's body being worked on by the medical professional.

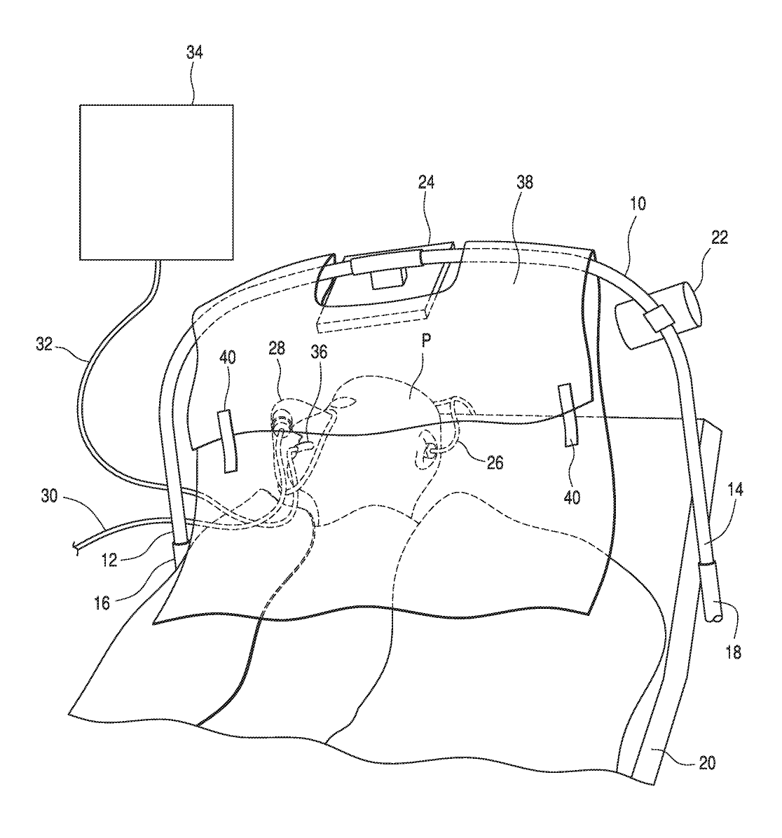

### CROSS-REFERENCE TO RELATED APPLICATIONS

The present application claims priority on prior U.S. Provisional Patent Application Ser. No. 62/105,859, filed Jan. 21, 2015, which is hereby incorporated herein in its entirety by reference.

### FIELD AND BACKGROUND OF THE INVENTION

The present invention is generally directed to medical surgery-related accessory devices, and more particularly to a method and system for reducing patient trauma or anxiety during a procedure or surgery.

Hundreds of medical procedures and surgeries are performed each day in hospitals, surgical centers, physician's offices, throughout the world. Although many procedures and surgeries require the patient to be entirely under the effect of anesthesia, a large number of procedures and surgeries only require a local anesthesia. In other words, the patient remains awake and fully conscious of the surroundings, while the procedure or surgery is being performed. While the recovery for a procedure or surgery performed under a local anesthesia is significantly faster, many patients experience trauma or anxiety as the procedure or surgery is being carried out. This is because the patient can observe and see the operation, as well as the medical personal around the patient's bed.

Currently, there is no device or system that eliminates or reduces patient trauma or anxiety during such surgery or procedure.

### ASPECTS AND SUMMARY OF THE INVENTION

The present disclosure is directed to various aspects of the present invention.

One aspect of the present invention is to provide a method and system that can be used by a medical professional to reduce patient trauma or anxiety during a surgery or procedure.

Another aspect of the present invention is to provide a method and system that can be quickly attached to the patient's bed during a procedure or surgery for creating a temporary separation between the area being operated on and the patient's upper body, particularly the head portion.

Another aspect of the present invention is to provide a method and system for reducing patient trauma or anxiety during a procedure or surgery that provides comfort to the patient by audio/video means.

Another aspect of the present invention is to provide a method and system for reducing patient trauma or anxiety during a procedure or surgery that reduces any fire hazard around the patient's face by transporting exhaled gases away from the patient's oxygen mask.

Another aspect of the present invention is to provide a method and system for reducing patient trauma or anxiety during a procedure or surgery that includes an air circulation means, such as a fan, for quick dissipation of sweat or perspiration on/around the patient's face.

In summary, the main aspect of the present invention is to provide a method or system that reduces patient anxiety or trauma during a procedure or surgery, particularly where the patient is under local anesthesia and is awake and conscious of the surroundings.

### BRIEF DESCRIPTION OF THE DRAWINGS

One of the above and other aspects, novel features and advantages of the present invention will become apparent from the following detailed description of a preferred embodiment(s) of the invention, as illustrated in the drawings, in which:

The FIGURE shows in perspective view a preferred embodiment of the device or system of the present invention in use.

### DETAILED DESCRIPTION OF THE PREFERRED EMBODIMENT(S) OF THE INVENTION

As illustrated in the FIGURE, the device or system, in accordance with the preferred embodiment of the present invention, includes a generally tubular, inverted U-shaped bracket 10, the left and right ends 12 and 14 of which are configured to be slidably received in corresponding yoke members 16 and 18, respectively, mounted on the sides of a patient bed 20. The bracket 10 can be easily removed by lifting upwardly so that the left and right ends 12 and 14 thereof are pulled out from the corresponding yoke members 16 and 18. A suitable fan 22 is mounted on one side of the bracket 10 to direct air towards the patient's face. A suitable monitor 24 is also attached to the bracket 10 to allow the patient to watch TV, video, etc. To provide audio, a suitable set of earphones 26 is included as part of the system. It is noted that the earphones 26 may also be used by the surgeon or other medical professional to communicate with the patient P during the procedure or surgery, or as necessary.

In the FIGURE, the reference numeral 28 indicates a suitable oxygen mask with a supply tube 30 connected to a source of oxygen (not shown). In order to prevent the patient exhaled gases from lingering in the area around the mask 28 and contaminating it, an exhale or scavenger tube 32 is added to the oxygen mask 28 that carries away the exhaled gases from the patient P to a suitable scavenger machine or unit 34. In order to facilitate the removal of the exhaled gases from the patient P, a mouthpiece 36 is connected to one end of the exhaled tube 32 and is held in place by the patient P during the procedure or surgery.

As further shown in the FIGURE, a suitable partition member 38 is draped over the bracket 10 and fastened on the opposite side by using suitable adhesive tapes or the like members 40 to secure in place. Once the patient's head area is partitioned off, the surgeon can then perform the procedure or surgery on the lower part of the patient's body, without creating any apprehension or anxiety to the patient P who would be conscious and aware of the surroundings.

It is noted herewith that although the system of the present invention is shown and described by separating the upper and lower regions of the patient P, the bracket 10 can be mounted differently on the patient bed 20 such that the patient's head area generally stays separate or partitioned off from the area where the surgeon performs the procedure or surgery.

While this invention has been described as having preferred sequences, ranges, ratios, steps, order of steps, materials, structures, symbols, indicia, graphics, color scheme(s), shapes, configurations, features, components, or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the claims appended hereto or presented later. The invention, therefore, is not limited to the preferred embodiment(s) shown/described herein.

What is claimed is:

1. A device for visually separating the portion of a patient's body being worked on by a medical professional during a surgery or procedure, comprising:
a) a bracket for mounting to the bed supporting a patient undergoing a surgery or procedure; and
b) a partition to be connected to said bracket for separating the portion of the patient's body being worked on a medical professional.

2. The device of claim 1, wherein:
a) said bracket is generally U-shaped and disposed in an inverted position over the patient.

3. The device of claim 2, further comprising:
a) a monitor attached to said bracket for providing a video feed for the patient.

4. The device of claim 2, further comprising:
a) an air circulation member attached to said bracket for circulating air about the patient.

5. The device of claim 2, further comprising:
a) means for communicating with the patient by the medical professional being visually separated by the device.

6. A device for reducing patient trauma or anxiety during a procedure or surgery, comprising:
a) a bracket for mounting to the bed supporting a patient undergoing a surgery or procedure;
b) a partition to be connected to said bracket for separating the portion of the patient's body being worked on a medical professional; and
c) wherein the device reduces trauma or anxiety to the patient by visually blocking the portion of the patient's body being worked on by the medical professional.

7. The device of claim 6, wherein:
a) said bracket is generally U-shaped and disposed in an inverted position over the patient.

8. The device of claim 7, further comprising:
a) a monitor attached to said bracket for providing a video feed for the patient.

9. The device of claim 7, further comprising:
a) an air circulation member attached to said bracket for circulating air about the patient.

10. The device of claim 7, further comprising:
a) means for communicating with the patient by the medical professional being visually separated by the device.
